(19) United States
(12) Patent Application Publication
(10) Pub. No.: US 2005/0187638 A1
(43) Pub. Date: Aug. 25, 2005

(54) Ceramic endoprosthesis components and processes for their production

(76) Inventors: Wilfried Glien, Klosterlausnitz (DE); Thomas Oberbach, Reichenbach (DE); Claudia Ortmann, Jena (DE)

Correspondence Address: GRAYBEAL, JACKSON, HALEY LLP, 155 - 108TH AVENUE NE, SUITE 350, BELLEVUE, WA, 98004-5901, US

(21) Appl. No.: 11/090,345

(22) Filed: Mar. 24, 2005

Related U.S. Application Data
(63) Continuation of application No. PCT/EP03/08286, filed on Jul. 28, 2003.

(30) Foreign Application Priority Data
Sep. 24, 2002 (DE) 10244439.0

(51) Int. Cl. A61F 2/28

(52) U.S. Cl. 623/23.56; 264/102; 264/122; 427/2.1

(57) ABSTRACT

The endoprosthesis component consists of a ceramic material which contains aluminium oxide and zirconium (di)oxide, the zirconium (di)oxide being present unstabilized or stabilized. The material has a gradient of the aluminium oxide and zirconium (di)oxide contents. In the area of increased tensile, bending and torsional stresses the zirconium (di)oxide content is increased compared with the articulation area, which contains predominantly aluminium oxide. The endoprosthesis component can be produced by infiltration of an open-pored pre-sintered aluminium oxide matrix by a zirconium (di)oxide slip, a zirconium salt solution, a zirconium-containing sol or alcoholate or a mixture of two or more of the abovementioned solutions/liquids. It can also be produced by continuously filling a compression mould with a mixture of aluminium oxide and zirconium (di)oxide powders, the mixing ratio of aluminium oxide and zirconium (di)oxide being varied during the filling of the compression mould.

### SUMMARY OF THE INVENTION

The invention relates to an endoprosthesis component made from a ceramic material which is essentially composed of aluminium oxide and zirconium (di)oxide, and processes for its production.

Aluminium oxide and zirconium (di)oxide have been known for years as proven ceramic implant materials. Aluminium oxide is very hard and resistant to wear. Zirconium (di)oxide is a very fracture-tough and damage-tolerant material. It is known that endoprosthesis components which in each case consist of one of the two materials are not freely combinable with each other in artificial joints, due to the frictional forces that occur. Pairings of joint partners made from aluminium oxide have proved successful, but material pairings of aluminium oxide with zirconium oxide and zirconium oxide with zirconium oxide are disputed in the literature, as very marked wear phenomena can result here.

EP 1 035 878 B1 presents possible material pairings for joint partners made from ceramic materials, through which novel structural configurations with optimum wear behaviour are to be made possible. The joint partners consist of sintered materials which are essentially aluminium oxide and zirconium oxide, at least one of the joint partners consisting of zirconium oxide with 0.1 to 40 wt. % added aluminium oxide. In one example a joint partner made from aluminium oxide is allocated to a joint partner made from zirconium oxide with an aluminium oxide content of over 5 wt. % and the material of the ball of a joint has a higher zirconium oxide content than the material of the ceramic socket insert.

It is known from WO 97/31592, in the case of an artificial hip joint, to make the socket from aluminium oxide and the capitulum from zirconium oxide.

A biomedical component is known from US 2002/0031675 A1 which consists of 90 mol.% zirconium dioxide, the zirconium dioxide being partially stabilized by at least 2.1 mol. %yttrium oxide, and containing between 0.05 and 1 wt. % aluminium oxide.

US 2002/0010070 A1 describes a biomedical component which consists of aluminium oxide reinforced with zirconium dioxide, the zirconium oxide content being 1 to 69 wt. % and the zirconium dioxide being stabilized by at least 2.1 mol. % yttrium oxide or rare earth oxides.

U.S. Pat. No. 6,312,473 B1 describes implant components which are covered with a layer of titanium or a titanium alloy. The open pores of this layer are impregnated with a biocompatible cement which is reinforced by selected oxides which contain aluminium oxide, magnesium oxide, zirconium oxide or a combination of these oxides.

U.S. Pat. No. 4,950,294 describes an implant component with a matrix made from aluminium oxide, zirconium (di)oxide and yttrium oxide. The surface of the matrix is not monocrystalline and a bioactive layer covers the matrix surface.

Endoprosthesis components made from a composite of aluminium oxide and zirconium (di)oxide are moreover known from EP 1 228 774 A1, EP 0 908 425 A1, JP 09268055 A and JP 11228221 A.

Hip-joint implants are known from WO 02/102275 in which the capitulum consists of a metal alloy and the joint socket of zirconium oxide and optionally with additions of aluminium oxide.

A process for the production of a porosity gradient for gradient materials is known from DE 44 35 146 C2, in which a porous body made from an electrically conductive material is immersed in an electrolyte of an electrolysis cell and an anodic removal of the material of the body is effected by application of an electrolyte current, the amount removed being variable along the connection line between anode and cathode.

The object of the invention is to create ceramic endoprosthesis components which are both hard and resistant to wear as well as fracture-tough and damage-tolerant.

According to the invention this object is achieved in that the material has a gradient of the aluminium oxide and zirconium (di)oxide contents.

The fact that the material has a gradient of the aluminium oxide and zirconium (di)oxide contents means that levels of these ceramic systems change along the gradient. The endoprosthesis component according to the invention does not therefore have a uniform material composition, but the aluminium oxide and zirconium (di)oxide contents vary within the endoprosthesis component. In contrast, the material composites made from zirconium oxide and aluminium oxide which are known from the printed documents named above have a homogeneous distribution of the components in the material.

The excellent wear properties of the aluminium oxide are to be useful above all in the areas of the endoprosthesis component in which ceramic sliding partners articulate against one another and the material is subjected to frictional stress. In these areas the aluminium oxide content is therefore higher and can be up to 100%. It is above all in the areas of the endoprosthesis component where stress maxima and surface pressures are to be expected that the properties of the zirconium (di)oxide are to be introduced. In these areas the zirconium oxide content is therefore higher and can be up to 100%.

0 to 0.3 wt. % magnesium oxide can be added as sintering auxiliary to the aluminium oxide, inhibiting grain growth. The zirconium (di)oxide can be present unstabilized or stabilized (with the additions known for phase stabilization of the rare earth oxides, alkaline-earth oxides, titanium oxide, chromium oxide or hafnium oxide).

The fracture resistance and fracture toughness and the damage tolerance of the ceramic endoprosthesis components can thereby be clearly enhanced. The material is able to degrade crack energy. If a crack meets a zirconium (di)oxide particle, this will result in crack branching and, if there is cubic or tetragonal modification of the zirconium (di)oxide, a phase transition into monoclinic zirconium (di)oxide, energy degradation taking place. The phase transition from tetragonal/cubic into monoclinic is associated with an increase in volume, with the result that the crack tip is compressed and crack growth is inhibited. This mechanism is known from the transition-reinforced ceramics available hitherto.

The endoprosthesis components according to the invention are a substance with a material and grain-size gradient which can be produced by two different processes.

In the first process, zirconium (di)oxide particles (particle size:<100 nm), in the case of stabilized zirconium (di)oxide including the stabilizers, are introduced by infiltration into a pre-sintered aluminium oxide endoprosthesis component (aluminium oxide matrix) which can contain 0 to 0.3 wt. % magnesium oxide and which possesses a high open porosity. The pre-sintering of the endoprosthesis component takes place at 800 to 1200° C., it being necessary to avoid shrinkage of the material, in order that the porosity is retained for infiltration. The production of the gradient in the material takes place using a process related to slip casting. The zirconium-containing sol (colloid-based or polymer-based; H. Richter, Herstellung keramischer Nanofiltrationsmembranen aus ZrO₂ und TiO₂ Dissertation, TU Bergakademie Freiberg, Faculty of Materials Science and Materials Technology, 1999), the zirconium salt solution, the zirconium alcoholate or the zirconium (di)oxide slip or also a mixture of two or more of the abovementioned solutions/liquids is applied (by e.g. pouring or spraying) to the pre-sintered, porous aluminium oxide matrix which can contain up to 0.3 wt. % magnesium oxide, or it is infiltrated. The zirconium-containing sol, the zirconium (di)oxide slip, the zirconium salt solution, the zirconium alcoholate or a mixture of two or more of the abovementioned solutions/liquids can contain the stabilizers already mentioned. Depending on the pore volume, the pore size, the time and the concentration of the liquid system, the porous material is infiltrated to a certain depth. The solid particles settle against the inner surface of the pores. After the infiltration the drying of the endoprosthesis component takes place, which must be done carefully in order that no cracks form in the material. This drying is followed by an outgassing process in order to eliminate the possibly present organic additives. These organic additives, which can be contained in the sol, slip, the salt solution or the alcoholate, must be removed before a subsequent sintering by thermal outgassing, as otherwise cracks and defects form in the material. So long as there is still sufficiently open porosity in the aluminium oxide matrix, the infiltration, drying and outgassing process can be repeated as often as desired. Any desired zirconium (di)oxide content can therefore be set in the aluminium oxide matrix. During the subsequent sintering at 1300 to 1600° C. a solid, graduated and dense (pore-free) material composite of aluminium oxide and zirconium (di)oxide (cubic, tetragonal and monoclinic phase) forms. On the side from which the infiltration takes place, up to 100% zirconium (di)oxide can be present. The concentration of the zirconium (di)oxide decreases continuously from the surface into the inside of the aluminium oxide matrix. A further compression can then take place by HIP (hot isostatic pressing).

The first process described can also be used such that aluminium oxide particles with 0 to 0.3 wt. % added magnesium oxide are incorporated into an unstabilized or stabilized (with the additions known for phase stabilization of the rare earth oxides, alkaline-earth oxides, titanium oxide, chromium oxide or hafnium oxide) zirconium (di)oxide matrix. The particles to be incorporated are present in the form of sols, slip, salt solutions, alcoholates or mixtures of two or more of the abovementioned solutions/liquids.

The matrix which is infiltrated can also be a homogeneous porous composite. The matrix can thus be aluminium oxide, zirconium (di)oxide or a composite. The composite can consist between 0 and 100% of aluminium oxide and correspondingly between 100 and 0% of zirconium (di)oxide. The zirconium (di)oxide can be an unstabilized zirconium (di)oxide or one stabilized with the usual stabilizers (rare earth oxides, alkaline-earth oxides, titanium oxide, chromium oxide or hafnium oxide—which are known as phase stabilizers).

In the second production process the gradient in the material is produced via uniaxial or isostatic dry pressing. The compression mould is continuously filled with zirconium (di)oxide and aluminium oxide powders, the mixing ratio of zirconium (di)oxide and aluminium oxide with up to 0.3 wt. % magnesium oxide being continuously varied depending on what is required and/or the design of the endoprosthesis component. A moulding is produced by subsequent pressing. All the mixing ratios of the main components zirconium (di)oxide (stabilized and unstabilized) and aluminium oxide (between 100% zirconium (di)oxide and 0% aluminium oxide and 100% aluminium oxide and 0% zirconium (di)oxide) can be realized. Areas of the ceramic component can also consist of aluminium oxide only, of zirconium (di)oxide only or of a composite of aluminium oxide and zirconium (di)oxide of homogeneous composition, other areas then consisting of a graduated material. The zirconium (di)oxide can again be unstabilized or stabilized material.

After the shaping, a thermal treatment is carried out in two steps. In the first step the organic material still present in the mouldings undergoes outgassing and in the second step the sintering of the mouldings takes place at 1300 to 1600° C. in order to achieve a solid, graduated and dense material composite of aluminium oxide and zirconium (di)oxide (monoclinic, tetragonal, cubic phase). Here too the moulding can be further compressed by HIP.

There is also the possibility that the produced moulding is subjected, after the outgassing, before or after a possible pre-sintering, to an infiltration process, corresponding to the first process described above.

In both production processes both phases (aluminium oxide and zirconium (di)oxide) in each case pass homogeneously into each other. The zirconium (di)oxide is present in tetragonal, cubic or monoclinic modification.

### BRIEF DESCRIPTION OF DRAWINGS

Embodiments of the ceramic endoprosthesis components according to the invention and the principle of the gradient production in the material are represented in the drawings and are described in more detail in the following. There are shown in:

FIG. 1 a ceramic hip capitulum in section,

FIG. 2 a ceramic socket of an artificial joint in section,

FIG. 3 a ceramic inlay of an artificial joint in section,

FIG. 4 a ceramic condyle skid of an artificial knee joint and

FIGS. 5 to 7 the principle of the production of the gradient in the material.

### DETAILED DESCRIPTION OF PREFERRED EMBODIMENTS

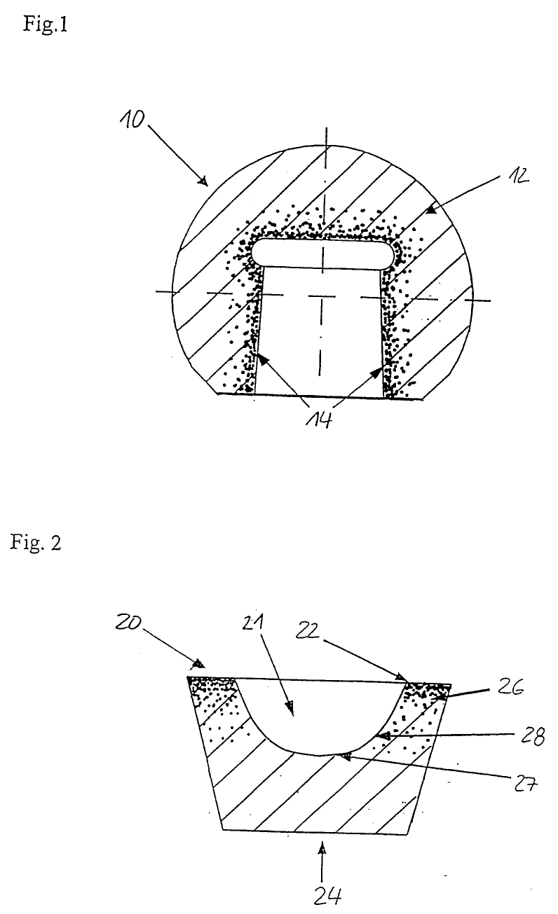

FIG. 1 shows a ceramic hip capitulum 10 that can be fitted onto a shaft of femur and is provided in order to articulate against a hip joint socket or an inlay in an artificial hip joint socket. The capitulum 10 consists of aluminium oxide and zirconium (di)oxide, the material having a concentration gradient and the aluminium oxide and zirconium (di)oxide contents changing along the gradient. The surface 12 of the capitulum 10 is subject to frictional stress and therefore has an increased aluminium oxide content. The surface 12 consists 100% of aluminium oxide. The hip capitulum 10 has an inner cone 14 by which it is fitted onto the shaft of femur. There are high surface pressures and stresses in circumferential direction in the inner cone 14 of the capitulum 10 after the hip head 10 has been fitted onto the shaft cone. Sudden and shock-like stresses can occur. A high zirconium (di)oxide content leads here to a higher fracture resistance, fracture toughness and damage tolerance than is the case with pure aluminium oxide. The surface of the inner cone 14 consists almost 100% of zirconium (di)oxide.

The production of the capitulum 10 with a gradient in the material takes place according to the first process described above. Firstly, an aluminium oxide capitulum 10 is produced and pre-sintered at 800 to 1300° C., with the result that a shrinkage of the material is avoided, in order that the porosity is retained for the infiltration. The aluminium oxide matrix contains 0.3 wt. % magnesium oxide and possesses a high open porosity. The zirconium (di)oxide slip, the zirconium-containing sol, the zirconium salt solution, the zirconium alcoholate or mixtures of two or more of the abovementioned solutions/liquids are poured into the inner cone areas 14. Zirconium-containing material including the stabilizers is incorporated by infiltration into the porous areas of the aluminium oxide matrix. Depending on the pore volume, the pore size, the time and the concentration of the liquid system, the porous capitulum 10 is infiltrated to the desired depth of 5 mm. The solid particles with the stabilizers settle against the inner surface of the pores. After the infiltration, the drying of the capitulum 10 takes place, which must be carried out carefully in order that no cracks form in the material. This drying is followed by an outgassing process for the elimination of the possibly present organic additives. The infiltration, drying and outgassing can be repeated several times, resulting in a zirconium (di)oxide content of almost 100% on the inner surface of the cone 14. During the subsequent sintering at 1300 to 1600° C., a solid, graduated and dense material composite of aluminium oxide and zirconium (di)oxide (cubic, tetragonal and monoclinic phase) forms. There is then a further densification of the capitulum 10 by HIP (hot isostatic pressing).

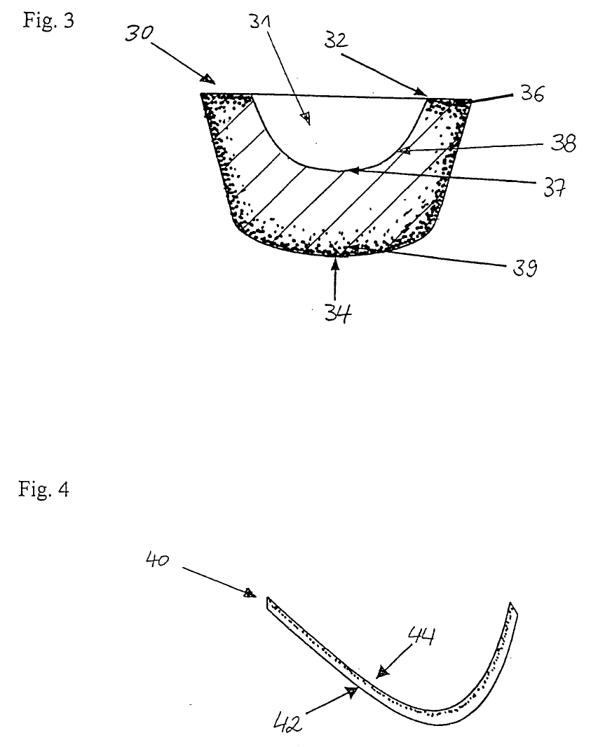

FIG. 2 shows a ceramic socket 20 and FIG. 3 shows a ceramic inlay 30, which are provided in order that a capitulum, e.g. the hip capitulum 10 described in FIG. 1, articulates against the insides 21, 31. Ceramic socket 20 and ceramic inlay 30 each possess a front surface and cap edge 22, 32 and an underside 24, 34. In the case of the ceramic socket 20 the upper area 26 has a zirconium (di)oxide content of almost 100% for the realization of a high damage tolerance in the case of impingement (striking of the shaft neck during movement), subluxation of the ceramic head and rim runners (contact between the ceramic head and the rim of the ceramic cup). In the area of the pole 27 of the cap and in the area of the main articulation 28 between ceramic head and cap the aluminium oxide content is 100% in order to guarantee a high resistance to wear. The ceramic inlay 30 is subject to stress in the upper area 36, like the ceramic socket 20, and therefore has a zirconium (di)oxide content of almost 100% in this area 36. In addition, the underside 34 of the inlay 30 is subject to tensile stresses, as the inlay 30 is fixed in a metal screw socket by conical clamping. Therefore a zirconium (di)oxide content of almost 100% is realized in the lower area 39. The zirconium (di)oxide content decreases continuously towards the pole 37 of the cap and towards the articulation area 38, as particularly good wear properties are required here, which are guaranteed by an aluminium oxide content of 100%.

The ceramic socket 20 and the gradient n the material are produced according to the second process described above: a compression mould required for the production of a ceramic socket 20 is continuously filled with aluminium oxide and zirconium (di)oxide powders, the mixing ratio of zirconium (di)oxide and aluminium oxide with 0.3 wt. % magnesium oxide being continuously varied. The filling of the compression mould begins in the areas which later form the underside 24 of the socket and continues in the direction of the front surface and cap edge 22. The compression mould is filled with aluminium oxide powder up to the height of what is later the main articulation area 28 of the cap. Upon further filling in the direction of the front surface and cap edge 22, a powder mixture of aluminium oxide and zirconium (di)oxide is then used, the zirconium (di)oxide powder content increasing continuously and then being almost 100% in the upper area 26 of the ceramic socket 20. The ceramic socket 20 is produced by uniaxial dry pressing and subsequent green machining, organic binders being added the while. After the shaping, a thermal treatment of the ceramic socket 20 is carried out in two steps. In the first step the organic material still present is gassed out and in the second step the sintering takes place at 1300 to 1600° C. in order to achieve a solid, graduated and dense material composite of aluminium oxide and zirconium (di)oxide (monoclinic, tetragonal, cubic phase), which can then be further compressed by HIP.

The ceramic inlay 30 and the gradient in the material are produced according to the first process described above. Firstly, a pre-sintered inlay is made from aluminium oxide according to the same principle as in the case of the capitulum in FIG. 1. Then a gradient is produced in the material by infiltration with zirconium (di)oxide including the known stabilizers. To this end, the inlay 30 is immersed in a zirconium-containing sol, a zirconium oxide salt solution, a zirconium alcoholate, a zirconium oxide slip or a mixture of the abovementioned solutions/liquids, with the result that the whole outer surface, except for the inside areas 31 which are provided for the articulation with a capitulum, is covered. The pre-sintered, porous aluminium oxide matrix can be infiltrated to a depth of approximately 5 mm. The infiltration is followed by the drying and outgassing. The infiltration, drying and outgassing can be repeated as often as desired, resulting in a zirconium (di)oxide content of almost 100% on the whole outer surface. The sintering then takes place at 1300 to 1600° C. A further densification by HIP is possible.

FIG. 4 shows a ceramic condyle skid 40 of an artificial knee joint which consists of aluminium oxide and zirconium (di)oxide, the levels of which change along a gradient. Through the introduction of zirconium (di)oxide, the fracture toughness and damage tolerance of the whole cup 40 is increased. The articulation surface (outside) 42, which is subject to frictional and wear stresses, consists 100% of aluminium oxide whilst the inside 44 of the cup consists almost 100% of zirconium (di)oxide in order to reduce rigidity and increase fracture toughness and damage tolerance.

The condyle skid 40 and the gradient in the material are produced according to the first process described above. Firstly, a condyle skid 40 is made from aluminium oxide and pre-sintered at 800 to 1300° C. Then a zirconium (di)oxide slip, a zirconium alcoholate, a zirconium-containing sol or a zirconium salt solution or a mixture of two or more of the abovementioned solutions/liquid is applied to the inside 44 of the cup 40 and the pre-sintered, porous aluminium oxide matrix is infiltrated from this side. The infiltration is followed by a drying and outgassing. A zirconium (di)oxide content of almost 100% results on the whole of the inside 44, which decreases continuously in the direction of the articulation surface (outside) 42. On the outside 42, the condyle skid 40 consists 100% of aluminium oxide. The condyle skid 40 is sintered at 1300 to 1600° C. and can then be densified by HIP.

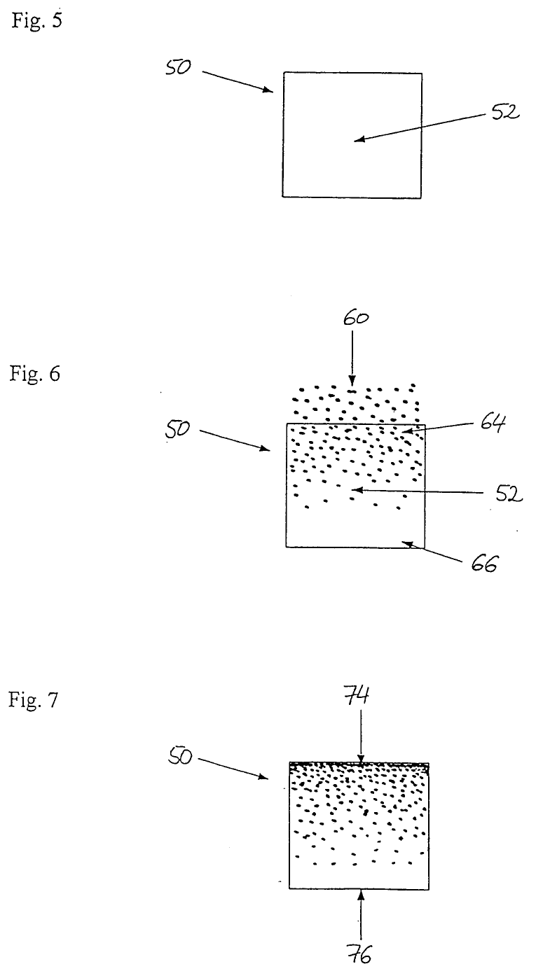

FIGS. 5 to 7 show diagrammatically the infiltration of an aluminium oxide matrix by zirconium-containing material for the production of a gradient in the material. A pre-sintered aluminium oxide endoprosthesis component 50 consists of an aluminium oxide matrix 52 with high open porosity, which can contain 0 to 0.3 wt. % magnesium oxide (FIG. 5). Zirconium-containing material 60 (particle size:<100 nm), in the case of stabilized zirconium (di)oxide including the stabilizers, is introduced by infiltration into the matrix 52 (FIG. 6). Depending on the pore volume, the pore size, the time and the concentration of the liquid system, the porous material is infiltrated to a specific depth. The zirconium-containing particles settle against the inner surface of the pores and the concentration of the zirconium (di)oxide decreases continuously from the surface 64 into the inside 66 of the aluminium oxide matrix. There can be up to 100% zirconium (di)oxide on the side 74 from which infiltration takes place, and 76 to 100% aluminium oxide on the opposite side (FIG. 7).

1-17. (canceled)

18. An endoprosthesis component comprising:
a ceramic material having an aluminium oxide component and a zirconium (di)oxide component, wherein the material comprises a gradient of the aluminium oxide and zirconium (di)oxide components.

19. The endoprosthesis component of claim 18, wherein the zirconium component is unstabilized.

20. The endoprosthesis component of claim 18, wherein the material further comprises at least one compound selected from the group comprising the rare earth oxides, alkaline-earth oxides, titanium oxide, chromium oxide and hafnium oxide, to stabilise the zirconium component.

21. The endoprosthesis component according to claim 18, which is part of an artificial joint having an articulation area and an area which when in use is subject to increased tensile, bending and torsional stresses, wherein the area of increased tensile, bending and torsional stresses comprises an increased zirconium (di)oxide content compared with the articulation area, and the articulation area contains predominantly aluminium oxide.

22. The endoprosthesis component according to claim 21, wherein the material has a mixture ratio between almost 100% aluminium oxide and 0% zirconium (di)oxide in the articulation area and a mixture ratio of 0% aluminium oxide and almost 100% zirconium (di)oxide in the area which when in use is subject to increased tensile, bending and torsional stresses.

23. The endoprosthesis component according to claim 18, wherein the material has a concentration gradient of aluminium oxide and zirconium (di)oxide.

24. The endoprosthesis component according to claim 23, wherein the material has a grain-size gradient, which is coupled to the concentration gradient.

25. The endoprosthesis component according to claim 18, wherein the aluminium oxide contains 0 to 0.3 wt. % magnesium oxide.

26. A process for producing an endoprosthesis component comprising a ceramic material having an aluminium oxide component and a zirconium (di)oxide component, wherein the material has a gradient of the aluminium oxide and zirconium (di)oxide components, the process comprising:
providing an endoprosthesis component comprising an open-pored, pre-sintered aluminium oxide matrix;
infiltrating the endoprosthesis component with zirconium containing particles from at least one of a zirconium (di)oxide slip, a zirconium salt solution, a zirconium-containing sol or alcoholate, or a mixture of two or more of the above-mentioned solutions/liquids, wherein particles are generally <100 nm and are introduced into the open-pored aluminium oxide matrix.

27. The process according to claim 26, wherein the endoprosthesis component comprises a matrix in the form of a homogeneous, porous composite of zirconium (di)oxide and aluminium oxide.

28. The process according to claim 26, wherein the infiltration takes place by at least one of spraying, immersion or pouring.

29. The process according to claim 26, wherein the infiltrated matrix of the endoprosthesis component is subjected, after the infiltration, to a drying, outgassing and sintering process.

30. The process according to claim 29, wherein the infiltrating, drying and outgassing process is repeated several times in order to increase the zirconium (di)oxide content in the matrix.

31. A process for producing an endoprosthesis component comprising a ceramic material having an aluminium oxide component and a zirconium (di)oxide component, wherein the material has a gradient of the aluminium oxide and zirconium (di)oxide components, the process comprising:
providing an endoprosthesis component comprising an open-pored, pre-sintered zirconium (di)oxide matrix stabilized with the addition of at least one of the rare earth oxides, alkaline-earth oxides, titanium oxide, chromium oxide or hafnium oxide or unstabilized; and
infiltrating the endoprosthesis component with aluminium oxide particles in the size range of <100 nm, the particles comprising from 0 to 0.3 wt. % magnesium oxide.

32. The process according to claim 31, wherein the endoprosthesis component has a matrix in the form of a homogeneous, porous composite of zirconium (di)oxide and aluminium oxide.

33. The process according to claim 31, wherein the infiltration takes place by at least one of spraying, immersion or pouring.

34. The process according to claim 31, wherein the infiltrated matrix of the endoprosthesis component is subjected, after the infiltration, to a drying, outgassing and sintering process.

35. The process according to claim 34, wherein the infiltrating, drying and out gassing process is repeated several times in order to increase the zirconium (di)oxide content in the matrix.

36. A process for producing an endoprosthesis component comprising a ceramic material having an aluminium oxide component and a zirconium (di)oxide component, wherein the material has a gradient of the aluminium oxide and zirconium (di)oxide components, the process comprising:
filling a compression mould at least partially continuously with a mixture of aluminium oxide and zirconium (di)oxide powders, the mixing ratio of aluminium oxide and zirconium (di)oxide being varied during the filling of the compression mould; and
producing a moulding by pressing.

37. The process according to claim 36, wherein the pressing is carried out uniaxially.

38. The process according to claim 36, wherein the is carried out isostatically.

39. The process according to claim 36, wherein the moulding is gassed out and sintered.

40. The process according to claim 36, further comprising:
gassing out the pressed moulding; and
infiltrating the moulding with zirconium containing particles from at least one of a zirconium (di)oxide slip, a zirconium salt solution, a zirconium-containing sol or alcoholate, or a mixture of two or more of the above-mentioned solutions/liquids, wherein the particles are generally <100 nm.

41. The process according to claim 40, further comprising densifying the moulding by hot isostatic pressing.

42. The process according to claim 36, further comprising:
gassing out the pressed moulding; and
infiltrating the endoprosthesis component with aluminium oxide particles in the size range of <100 nm, the particles comprising from 0 to 0.3 wt. % magnesium oxide.

43. The process according to claim 42, comprising densifying the moulding by hot isostatic pressing.
